(19) United States
(12) Patent Application Publication
(10) Pub. No.: US 2016/0349216 A1
(43) Pub. Date: Dec. 1, 2016

(54) ODOR SENSORS

(71) Applicant: EMPIRE TECHNOLOGY DEVELOPMENT LLC, Wilmington, DE (US)

(72) Inventors: Naoya Ichimura, Osaka (JP); Tatsuaki Hirase, Osaka (JP)

(73) Assignee: EMPIRE TECHNOLOGY DEVELOPMENT LLC, Wilmington, DE (US)

(21) Appl. No.: 15/115,012

(22) PCT Filed: Jan. 30, 2014
(86) PCT No.: PCT/US14/13871

§ 371 Date: Jul. 28, 2016

(51) Int. Cl. G01N 29/02; H03H 3/02; H03H 9/19; G01N 33/00; G01N 29/036

(52) U.S. Cl. CPC G01N 29/022; G01N 33/0031; G01N 29/036; G01N 33/0047; H03H 9/19; H03H 3/02; G01N 2291/0426; G01N 2291/021

(57) ABSTRACT

Odor sensors and methods for their preparation and use are described. In some examples, an odor sensor may include a convex quartz crystal resonator having a first surface and a second surface, a pair of first electrodes disposed on the first surface, a second electrode disposed on the second surface, and at least one odor-sensitive material disposed on the second electrode.

### BACKGROUND

Unless otherwise indicated herein, the materials described herein are not prior art to the claims in the present application and are not admitted to be prior art by inclusion in this section.

Odor is produced by volatile chemical compounds. A variety of sensors, including a chemical sensor, a biosensor, a mass spectrometer, a differential optical absorption spectrometer, etc., are available for detecting and identifying odor.

Quartz possesses piezoelectric properties. In this regard, a resonant frequency of a quartz crystal resonator is changed when a mass change is made to the resonator. For instance, when an odorant (that is, a chemical causing an odor) is adhered or adsorbed to the surface of the resonator, the resonant frequency of the resonator is changed. Thus, the quartz crystal resonator can be employed as an odor sensor based on the resonant frequency change due to the adhesion/adsorption of the odorant.

Conventionally, a disk-shaped quartz crystal resonator has been employed as an odor sensor. The disk-shaped resonator is suspended in air so that the entire resonator can be physically vibrated. However, the vibration of the entire resonator harms the stability of the resonant frequency change in terms of the adhesion/adsorption of the odorant.

### SUMMARY

Some embodiments disclosed herein include an odor sensor including a convex quartz crystal resonator having a first surface and a second surface, a pair of first electrodes disposed on the first surface, a second electrode disposed on the second surface, and at least one odor-sensitive material disposed on the second electrode. In some embodiments, the convex quartz crystal resonator may be a plano-convex quartz crystal resonator. In the embodiments, the first surface may be a convex-shaped surface, and the second surface may be a planar surface. In some embodiments, the convex quartz crystal resonator may be an AT-cut convex quartz crystal resonator.

In some embodiments, the first electrodes and the second electrode may be made of gold. In some embodiments, the pair of first electrodes and the second electrode may be aligned with a convex-shaped portion of the convex quartz crystal resonator.

In some embodiments, the odor-sensitive material may have a selective affinity for a chemical to be detected. By way of example, but not limitation, the odor-sensitive material may include at least one of polycaprolactone, polystyrene, cycloolefin, and acrylic resin.

In some embodiments, the at least one odor-sensitive material may be applied on the second electrode by applying on the second electrode a solution including the at least one odor-sensitive material. In some embodiments, the solution may include an organic solvent that may dissolve the at least one odor-sensitive material. By way of example, but not limitation, the organic solvent may include at least one of acetone, trichloroethylene, and alcohol.

Also provided is a method for detecting odor using an odor sensor including any of the odor sensors provided herein.

Alternative embodiments disclosed herein may include a method for fabricating an odor sensor. In some embodiments, the method may include providing a quartz crystal substrate, forming a convex portion in the quartz crystal substrate, forming a pair of first electrodes on a first surface of the convex portion, forming a second electrode on a second surface of the convex portion, and applying at least one odor-sensitive material on the second electrode.

In some embodiments, the convex portion may be formed by applying a photoresist on a surface of the quartz crystal substrate, patterning the photoresist on the surface of the quartz crystal substrate, curing the patterned photoresist, and etching the quartz crystal substrate and the patterned photoresist. In some embodiments, the convex portion may be formed further by determining a sectional profile of the convex portion, and patterning the photoresist based at least in part on the determined sectional profile of the convex portion. In some embodiments, the patterned photoresist may be cured by heating the patterned photoresist. In some embodiments, the quartz crystal substrate and the patterned photoresist may be etched by reactive ion etching (ME). In some embodiments, the quartz crystal substrate and the patterned photoresist may be etched at different etching rates.

In some embodiments, the pair of first electrodes may be formed by sputtering gold on the first surface of the convex portion, and patterning the sputtered gold. In some embodiments, the second electrode may be formed by sputtering gold on the second surface of the convex portion, and patterning the sputtered gold.

In some embodiments, the method may further include selecting the at least one odor-sensitive material to be applied based at least in part on a chemical to be detected. In some embodiments, the method may further include selecting an amount of the at least one odor-sensitive material to be applied on the second electrode. In some embodiments, the method may further include selecting an area of the second electrode on which the at least one odor-sensitive material to be applied.

Also provided is an odor sensor fabricated by any of the methods provided herein.

Also provided is a method for detecting odor using an odor sensor fabricated by any of the methods provided herein.

Yet alternative embodiments disclosed herein may include a method for detecting odor using an odor sensor including at least one convex quartz crystal resonator with at least one odor-sensitive material disposed thereon. In some embodiments, the method may include measuring a change in resonating frequency of the at least one convex quartz crystal resonator, and detecting a chemical associated with the at least one odor-sensitive material based at least in part on the measured change in resonating frequency of the at least one convex quartz crystal resonator.

In some embodiments, each convex quartz crystal resonator may have a first surface and a second surface, a pair of first electrodes may be disposed on the first surface, a second electrode may be disposed on the second surface, and the at least one odor-sensitive material may be disposed on the second electrode.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1A is a schematic sectional view of an illustrative example of an odor sensor, arranged in accordance with at least some embodiments described herein;

FIG. 1B is a schematic top view of the illustrative example of the odor sensor shown in FIG. 1A;

FIG. 1C is a schematic bottom view of the illustrative example of the odor sensor shown in FIG. 1A;

FIG. 2A is a schematic sectional view of an illustrative example of an odor sensor having an array of sensor elements, arranged in accordance with at least some embodiments described herein;

FIG. 2B is a schematic top view of the illustrative example of the odor sensor shown in FIG. 2A;

FIG. 2C is a schematic bottom view of the illustrative example of the odor sensor shown in FIG. 2A;

FIG. 3 schematically shows an illustrative example of an oscillation circuit, arranged in accordance with at least some embodiments described herein;

FIG. 4 illustrates an example flow diagram of a process for fabricating an odor sensor, arranged in accordance with at least some embodiments described herein;

FIG. 5 illustrates an example flow diagram of a process for detecting odor using an odor sensor, arranged in accordance with at least some embodiments described herein;

FIGS. 6A-6E show examples of resonant frequency changes, arranged in accordance with at least some embodiments described herein; and

FIG. 7 shows examples of resonant frequency changes, arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the drawings, can be arranged, substituted, combined, separated, and designed in a wide variety of different configurations, all of which are explicitly contemplated herein.

Technologies are herein generally described for an odor sensor.

In some examples, the odor sensor may include a convex quartz crystal resonator, a pair of first electrodes disposed on a first surface of the convex quartz crystal resonator, a second electrode disposed on a second surface of the convex quartz crystal resonator, and least one odor-sensitive material disposed on the second electrode. The odor-sensitive material may have a selective affinity for a chemical to be detected.

In some examples, the odor sensor may include at least one convex quartz crystal resonator, on each of which at least one odor-sensitive material may be disposed. The odor sensor may enable to measure a change in resonating frequency of the at least one convex quartz crystal resonator, and detect a chemical associated with the at least one odor-sensitive material based at least in part on the measured change in resonating frequency.

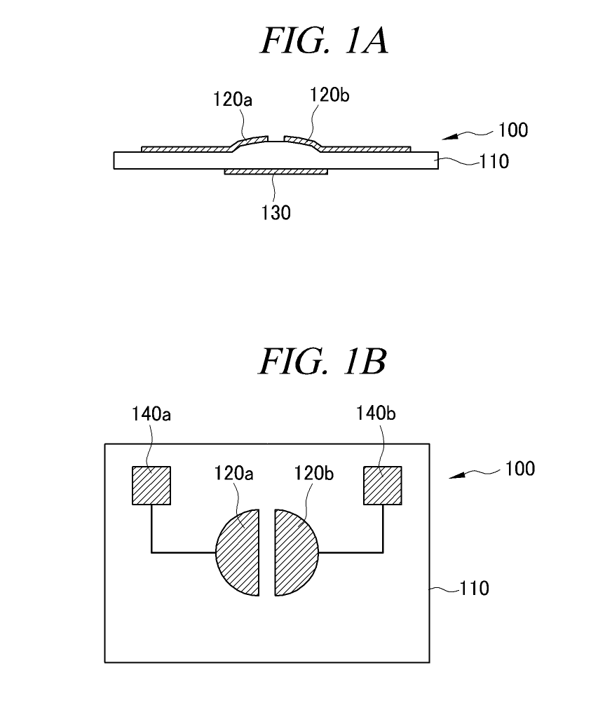

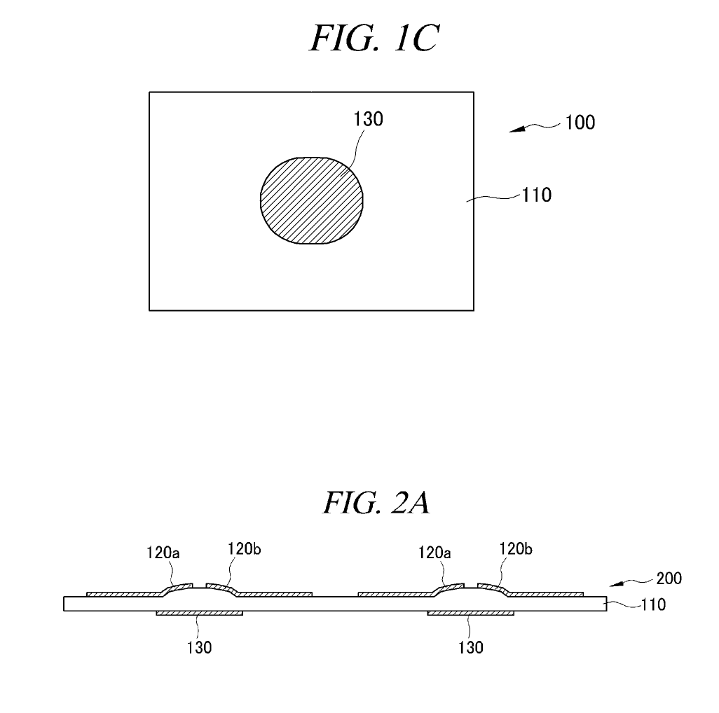

FIGS. 1A-1C schematically show an illustrative example of an odor sensor, arranged in accordance with at least some embodiments described herein. FIGS. 1A-1C are a schematic sectional view, a schematic top view and a schematic bottom view of the example odor sensor, respectively.

As depicted in FIGS. 1A-1C, an odor sensor 100 may include a convex quartz crystal resonator 110, a pair of first electrodes 120a and 120b, and a second electrode 130. In some embodiments, at least one odor-sensitive material may be disposed on second electrode 130.

In some embodiments, convex quartz crystal resonator 110 may be a plano-convex quartz crystal resonator, which may have a convex-shaped portion and a non-convex-shaped portion, so that the convex-shaped portion may vibrate, while the non-convex-shaped portion may not. By way of example, but not limitation, convex quartz crystal resonator 110 may have a rectangular shape with a dimension of about 5 mm×5 mm from a top view, and the convex-shaped portion may have a circular shape with a diameter of about 1 mm to about 2 mm also from a top view. The shapes and/or dimensions of convex quartz crystal resonator 110 and the convex-shaped portion may vary depending on the desired implementation. By way of example, but not limitation, a thickness of the non-convex-shaped portion may be about 5 μm to about 2100 μm. Specific examples of thicknesses include about 5 μm, about 10 μm, about 50 μm, about 100 μm, about 500 μm, about 1000 μm, about 1500 μm, about 2000 μm, about 2100 μm, and ranges between any two of these values (including endpoints). The convex-shaped portion may be protruded from the surface of the non-convex shaped portion. The distance of this protrusion can be, for example, about 0.003 μm to about 30 μm, about 0.03 μm to about 30 μm, about 0.3 μm to about 30 μm, or about 3 μm to about 30 μm. Specific examples of the distance include about 0.003 μm, about 0.03 μm, about 0.3 μm, about 3 μm, about 10 μm, about 20 μm, about 30 μm, and ranges between any two of these values (including endpoints).

In some embodiments, convex quartz crystal resonator 110 may be a plano-convex quartz crystal resonator having a convex-shaped surface and a planar surface. In such cases, first electrodes 120a and 120b may be disposed on the convex-shaped surface, while second electrode 130 may be disposed on the planar surface.

In some embodiments, first electrodes 120a and 120b may be made of a conductive material such as, for example, gold, platinum, titanium, chromium, aluminum, nickel, silver, or any combination thereof. Second electrode 130 may also be made of a conductive material such as, for example, gold, platinum, titanium, chromium, aluminum, nickel, silver, or any combination thereof.

In some embodiments, first electrodes 120a, 120b and second electrode 130 may be aligned with the convex-shaped portion of convex quartz crystal resonator 110, so that first electrodes 120a, 120b may cover at least a part of the convex-shaped portion and second electrode 130 may also do. By way of example, but not limitation, a space between first electrodes 120a and 120b may be about 1 to 3 times of the non-convex-shaped portion of convex quartz crystal resonator 110. That is, the space between first electrodes 120a and 120b may be about 0.1 μm to about 3000 μm. Specific examples of thicknesses include about 0.1 μm, about 1 μm, about 10 μm, about 100 μm, about 500 μm, about 1000 μm, about 2000 μm, about 3000 μm, and ranges between any two of these values (including endpoints). By way of example, but not limitation, thicknesses of first electrodes 120a, 120b and second electrode 130 may be about 0.001 μm to about 1 μm. Specific examples of thicknesses include about 0.001 μm, about 0.01 μm, about 0.1 μm, about 1 μm, and ranges between any two of these values (including endpoints).

In some embodiments, odor sensor 100 may further include wiring pads 140a and 140b, respectively connected to first electrodes 120a and 120b. Wiring pads 140a and 140b may be disposed on the non-convex-shaped portion of convex quartz crystal resonator 110. By way of example, but not limitation, wiring pads 140a and 140b may be made of the same conductive material as first electrodes 120a and 120b, such as, for example, gold, platinum, titanium, chromium, aluminum, nickel, silver, or any combination thereof.

In some embodiments, the odor-sensitive material may have a selective affinity for a chemical to be detected. By way of example, but not limitation, the odor-sensitive material may include at least one of polycaprolactone, polystyrene, cycloolefin, or acrylic resin. For instance, polycaprolactone may detect phenylethyl alcohol (with a rose-like odor) but may not detect trichloroethylene, while polystyrene may detect both phenylethyl alcohol and trichloroethylene.

In some embodiments, an amount of the at least one odor-sensitive material and/or an area of second electrode 130 on which the at least one odor-sensitive material to be applied may vary depending on the desired implementation. The odor-sensitive material may be coated on the entire surface of the second electrode 130. Alternatively, an amount of the odor-sensitive material may be applied on a specific area such as a center of the second electrode 130 first, then the material may be applied repeatedly on that area to form a coating of desired thickness/shape to increase the sensitivity.

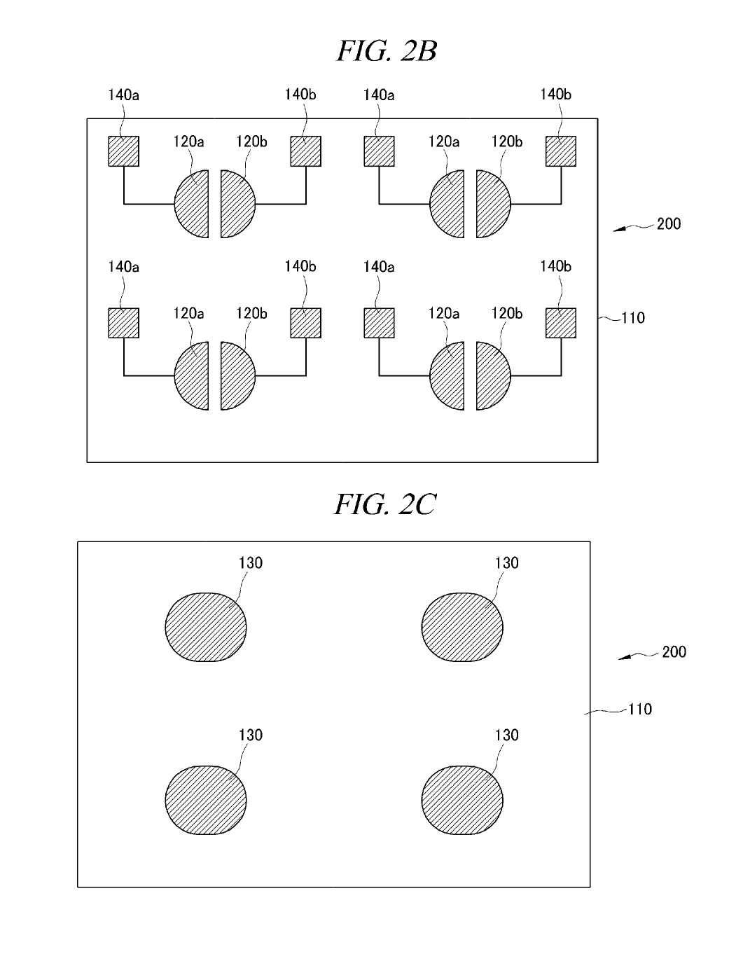

FIGS. 2A-2C schematically show an illustrative example of an odor sensor having an array of sensor elements, arranged in accordance with at least some embodiments described herein. FIGS. 2A-2C are a schematic sectional view, a schematic top view and a schematic bottom view of the example odor sensor, respectively.

As depicted in FIGS. 2A-2C, an odor sensor 200 may include 2×2 sensor elements, each of which may include convex quartz crystal resonator 110, first electrodes 120a and 120b, second electrode 130, and wiring pads 140a and 140b. That is, each sensor element may correspond to odor sensor 100 as illustrated in FIGS. 1A-1C. In some embodiments, the shapes and/or dimensions of convex quartz crystal resonator 110, first electrodes 120a and 120b, second electrode 130, and wiring pads 140a and 140b may be different in each sensor element.

In some embodiments, at least one odor-sensitive material may be disposed each second electrode 130. The disposed at least one odor-sensitive material may be different in each sensor element. Further, an amount of the at least one odor-sensitive material and/or an area of second electrode 130 on which the at least one odor-sensitive material is applied may also be different in each sensor element.

In some embodiments, odor sensor 200 may detect multiple odors simultaneously based on each resonant frequency change in each sensor element.

Although FIGS. 2A-2C illustrate that odor sensor 200 has 2×2 sensor elements, those skilled in the art will recognize that odor sensor 200 may include any number and/or arrangement of sensor elements.

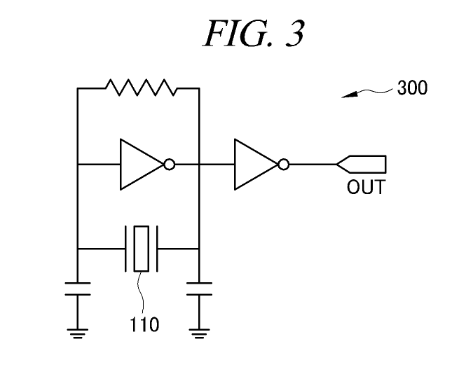

FIG. 3 schematically shows an illustrative example of an oscillation circuit, arranged in accordance with at least some embodiments described herein.

As depicted, an oscillation circuit 300 may include convex quartz crystal resonator 110. In some embodiments, convex quartz crystal resonator 110 may be connected other elements of oscillation circuit 300 via wiring pads 140a and 140b. An output terminal (OUT) of oscillation circuit 300 may be coupled with a frequency counter (not shown), so that the frequency counter may measure a resonating frequency of convex quartz crystal resonator 110. A change in the resonating frequency may be used to detect a chemical associated with at least one odor-sensitive material disposed on convex quartz crystal resonator 110.

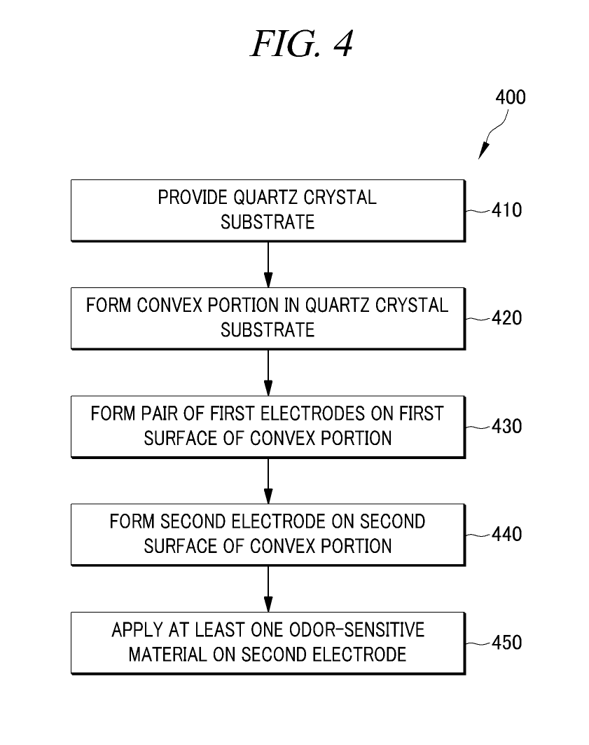

FIG. 4 illustrates an example flow diagram of a process for fabricating an odor sensor, arranged in accordance with at least some embodiments described herein.

An example process 400 may include one or more operations, actions, or functions as illustrated by one or more blocks 410, 420, 430, 440 and/or 450. Although illustrated as discrete blocks, various blocks may be divided into additional blocks, combined into fewer blocks, or eliminated, depending on the desired implementation.

At block 410, a quartz crystal substrate may be provided. By way of example, but not limitation, the quartz crystal substrate may be an AT-cut quartz crystal substrate.

At block 420, a convex portion (for example, convex quartz crystal resonator 110 in FIGS. 1-2) may be formed in the quartz crystal substrate. In some embodiments, the convex portion may be formed by applying a photoresist on a surface of the quartz crystal substrate, patterning the photoresist on the surface of the quartz crystal substrate, curing the patterned photoresist, and etching the quartz crystal substrate and the patterned photoresist at different etching rates.

In some embodiments, a sectional profile of the convex portion may be determined before the patterning of the photoresist. In such cases, the patterning of the photoresist may be performed based at least in part on the determined sectional profile of the convex portion. Further, in some embodiments, the curing of the patterned photoresist may be performed by heating the patterned photoresist. Further, in some embodiments, the etching may be performed by reactive ion etching (ME).

At block 430, a pair of first electrodes (for example, first electrodes 120a and 120b in FIGS. 1-2) may be formed on a first surface of the convex portion. In some embodiments, the first surface of the convex portion may be a convex-shaped surface of the convex portion. In some embodiments, the pair of first electrodes may be formed by sputtering a conductive material (for example, gold, platinum, titanium, chromium, aluminum, nickel, silver, or any combination thereof) on the first surface of the convex portion, and patterning the sputtered conductive material by wet etching for example.

At block 440, a second electrode (for example, second electrode 130 in FIGS. 1-2) may be formed on a second surface of the convex portion. In some embodiments, the second surface may be a planar surface of the convex portion. In some embodiments, the second electrode may be formed by sputtering a conductive material (for example, gold, platinum, titanium, chromium, aluminum, nickel, silver, or any combination thereof) on the second surface of the convex portion, and patterning the sputtered conductive material.

At block 450, at least one odor-sensitive material may be applied on the second electrode. The odor-sensitive material may be a material having a selective affinity for a chemical to be detected, such as polycaprolactone, polystyrene, cycloolefin, acrylic resin, and so on.

In some embodiments, a solution including the at least one odor-sensitive material may be applied on the second electrode. By way of example, but not limitation, the solution may include an organic solvent that may dissolve the at least one odor-sensitive material such as, for example, acetone, trichloroethylene, alcohol, or any combination thereof.

In some embodiments, an amount of the at least one odor-sensitive material to be applied on the second electrode, an area of the second electrode on which the at least one odor-sensitive material to be applied, an amount of the solution including the at least one odor-sensitive material to be applied on the second electrode, and/or a concentration of the at least one odor-sensitive material in the solution to be applied on the second electrode may vary depending on the desired implementation.

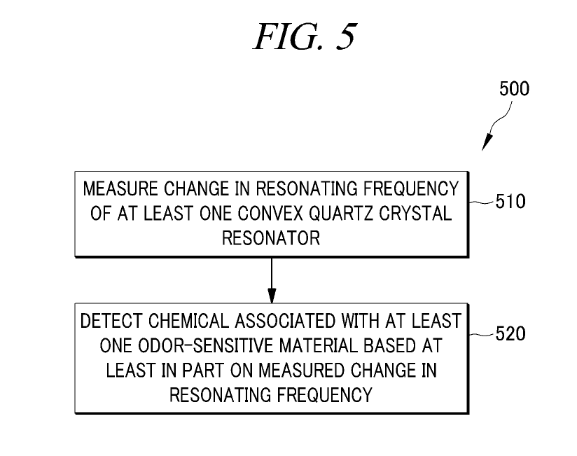

FIG. 5 illustrates an example flow diagram of a process for detecting odor using an odor sensor, arranged in accordance with at least some embodiments described herein.

An example process 500 may be performed by an odor sensor (for example, odor sensor 100 in FIG. 1 or odor sensor 200 in FIG. 2) including at least one convex quartz crystal resonator (for example, convex quartz crystal resonator 110 in FIGS. 1-2) with at least one odor-sensitive material disposed thereon. Process 500 may include one or more operations, actions, or functions as illustrated by one or more blocks 510 and/or 520. Although illustrated as discrete blocks, various blocks may be divided into additional blocks, combined into fewer blocks, or eliminated, depending on the desired implementation.

At block 510, a change in resonating frequency of the at least one convex quartz crystal resonator of the odor sensor may be measured. In some embodiments, the resonating frequency of the respective one of the at least one convex quartz crystal resonator may be measured by a frequency counter operatively coupled to the corresponding convex quartz crystal resonator.

At block 520, a chemical associated with the at least one odor-sensitive material may be detected based at least in part on the measured change in resonating frequency of the at least one convex quartz crystal resonator.

One skilled in the art will appreciate that, these and other processes and methods disclosed herein may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

By way of example, but not limitation, an electronic device such as, for example, a smartphone, a mobile phone, a personal digital assistant (PDA), a tablet, a laptop computer, a desktop computer, a television, a game console, etc. may be equipped with the example odor sensors described herein.

### EXAMPLES

The present disclosure will be understood more readily by reference to the following examples, which are provided by way of illustration and are not intended to be limiting in any way.

### Example 1

### Preparation of Convex Quartz Crystal Resonator

A convex quartz crystal resonator was prepared by applying a photoresist on a surface of an AT-cut quartz crystal substrate with a thickness of 100 μm, patterning the photoresist on the surface of the quartz crystal substrate, heat-curing the patterned photoresist, and etching the quartz crystal substrate and the patterned photoresist by reactive ion etching (ME). Then, gold was sputtered on a convex-shaped surface of the convex quartz crystal resonator, and the sputtered gold was patterned to form a pair of first electrodes and wiring pads. Further, gold was sputtered on a planar surface of the convex quartz crystal resonator, and the sputtered gold was patterned to form a second electrode.

### Example 2

### Preparation of Disk-Shaped Quartz Crystal Resonator as Comparative Example

A disk-shaped AT-cut quartz crystal resonator with a diameter of 0.5 cm was prepared. A natural frequency of the disk-shaped quartz crystal resonator was about 27 MHz. Electrodes made of gold were formed on both surfaces of the disk-shaped quartz crystal resonator.

### Example 3

### Preparation of Convex Quartz Crystal Resonator Coated with Odor-Sensitive Material

Polycaprolactone was used as an odor-sensitive material. A solution in which 50 ng of polycaprolactone was dissolved in trichloroethylene with a concentration of 60 ng/μL was applied on the second electrode disposed on the planar surface of the convex quartz crystal resonator (prepared above in Example 1) to form a convex quartz crystal resonator coated with polycaprolactone. Polycaprolactone was uniformly disposed and coated on the second electrode, and the thickness of the coating was 14 nm.

A general purpose electric circuit simulation was performed for the convex quartz crystal resonator coated with polycaprolactone to measure a resonating frequency of the convex quartz crystal resonator coated with polycaprolactone. The measured resonating frequency was about 17 MHz.

### Example 4

### Preparation of Oscillation Circuit

An oscillation circuit including a hex inverter TC74HCU04AP (produced by Toshiba Semiconductor) was prepared. A universal frequency counter/timer 53131A (produced by Agilent Technologies) was connected to an output terminal of the oscillation circuit via a coaxial cable. Resonating frequencies measured by the universal frequency counter/timer were collected by a computer via a GPIB (General Purpose Interface Bus) at every one second.

The wiring pads of the convex quartz crystal resonator coated with polycaprolactone (prepared above in Example 3) were connected to other elements of the oscillation circuit using a conductive adhesive. Using the oscillation circuit, the resonating frequency of the convex quartz crystal resonator coated with polycaprolactone was measured. The measured resonating frequency was about 16.7 MHz.

### Example 5

### Resonant Frequency Change in Response to Odorant

The convex quartz crystal resonator coated with polycaprolactone (prepared above in Example 3) was enclosed in a glass chamber. The glass chamber was a lab environmental chamber with a pressure of 1 atm, with a temperature of 25 degrees Celsius, and with a humidity of 40%. Respective one of sample odorants was further introduced to the glass chamber by applying 1 μL of a solution, in which the respective one of sample odorants was diluted at a ratio of 1:100 in acetone, to a tip of a cotton swab, and putting the cotton swab into the glass chamber. Then, the resonating frequency was measured at every one second by the oscillation circuit (prepared above in Example 4). As a comparative example, the resonating frequency was also measured under the condition in which 1 μL of acetone was applied to a tip of a cotton swab, and the cotton swab with acetone was put into the glass chamber. The sample odorants were phenylethyl alcohol (with a rose-like odor), methylcyclopentenolone (with a caramel-like odor), undecalactone (with a peach-like odor) and trichloroethylene (with a chloroform-like odor).

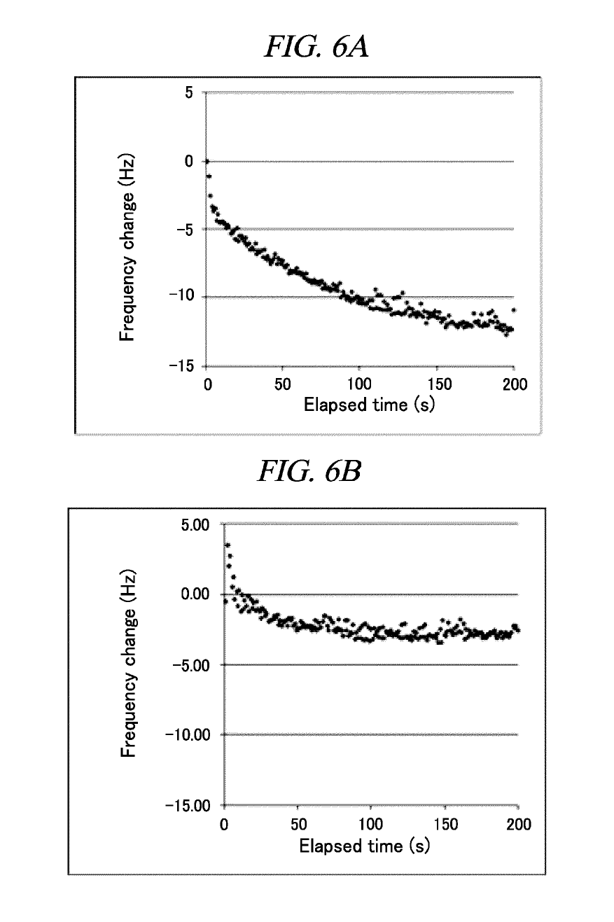

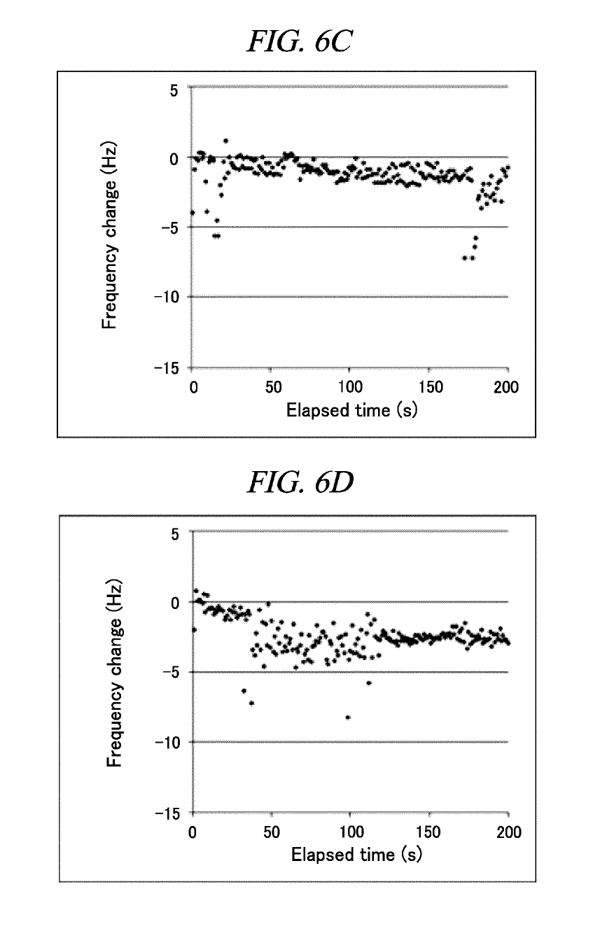

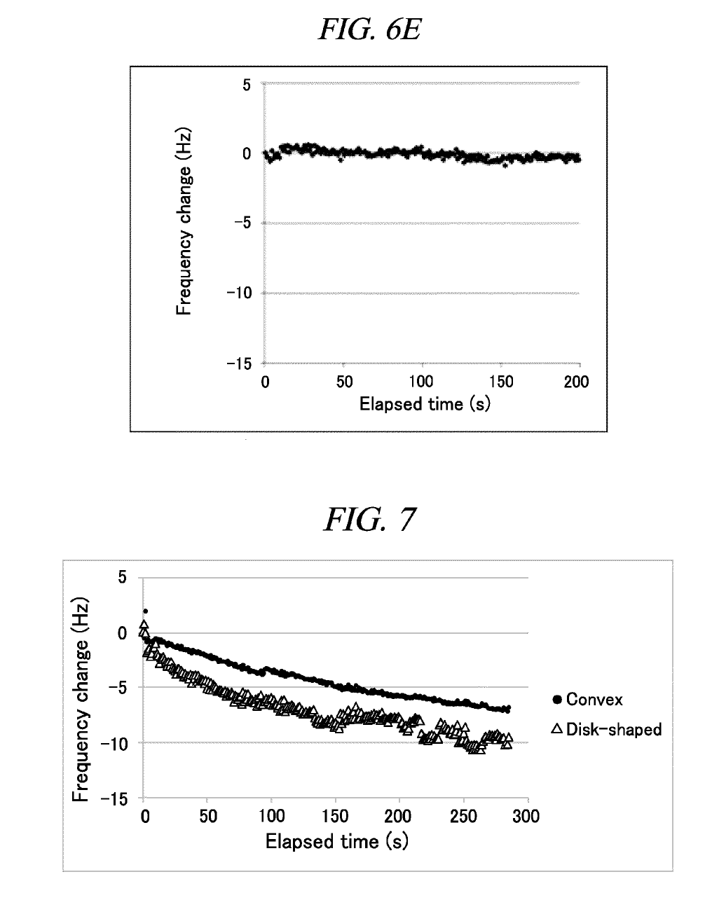

FIGS. 6A-6E show resonant frequency changes of the convex quartz crystal resonator coated with polycaprolactone, respectively measured under the condition in which phenylethyl alcohol (rose-like odor) was introduced, methylcyclopentenolone (caramel-like odor) was introduced, undecalactone (peach-like odor) was introduced, trichloroethylene (chloroform-like odor) was introduced, and acetone was introduced to the glass chamber. As shown, the resonant frequency was changed when each of the odorants was introduced to the glass chamber. As such, the convex quartz crystal resonator coated with polycaprolactone acted as an odor sensor.

### Example 6

### Comparison between Resonant Frequency Changes of Convex Quartz Crystal Resonator and Disk-Shaped Quartz Crystal Resonator

A solution in which 10 ng of polycaprolactone was dissolved in trichloroethylene with a concentration of 10 ng/μL was applied on the second electrode disposed on the planar surface of the convex quartz crystal resonator (prepared above in Example 1) to form a convex quartz crystal resonator coated with polycaprolactone. Polycaprolactone was uniformly disposed and coated on the second electrode, and the thickness of the coating was 3 nm. Further, 10 ng of polycaprolactone was also applied on one of the electrodes of the disk-shaped quartz crystal resonator (prepared above in Example 2) to form a disk-shaped quartz crystal resonator coated with polycaprolactone. The convex quartz crystal resonator coated with polycaprolactone and the disk-shaped quartz crystal resonator coated with polycaprolactone were enclosed in a glass chamber. The glass chamber was a lab environmental chamber with a pressure of 1 atm, with a temperature of 25 degrees Celsius, and with a humidity of 40%.

A solution, in which phenylethyl alcohol (rose-like odor) was diluted at a ratio of 1:30 in acetone, was prepared. 1 μL of the solution was applied to a cotton swab, and the cotton swab was put into the glass chamber. Then, the resonating frequency was measured at every one second by the oscillation circuit (prepared above in Example 4) for the two resonators.

FIG. 7 shows resonant frequency changes of the convex quartz crystal resonator coated with polycaprolactone and the disk-shaped quartz crystal resonator coated with polycaprolactone. As shown, the resonant frequency change in the convex quartz crystal resonator was more stable than the resonant frequency change in the disk-shaped quartz crystal resonator. This is because, at least, a portion (that is, convex-shaped portion) of the convex quartz crystal resonator was physically vibrated, while the entire disk-shaped quartz crystal resonator was physically vibrated, which resulted in complex oscillation modes, and thus loss of vibration energy.

Further, it was shown that the response sensitivity of the disk-shaped quartz crystal resonator with the resonating frequency of about 27 MHz was only about 1.7 time of the response sensitivity of the convex quartz crystal resonator with the resonating frequency of about 17 MHz, although it is theoretically expected that the response sensitivity of the disk-shaped quartz crystal resonator with the resonating frequency of 27 MHz is about 2.5 (=27²/17²) times of the response sensitivity of the disk-shaped quartz crystal resonator with the resonating frequency of 17 MHz. This means that the response sensitivity of the convex quartz crystal resonator is better than the response sensitivity of the disk-shaped quartz crystal resonator under the condition of the same resonating frequency. This is also because, at least, a portion (that is, convex-shaped portion) of the convex quartz crystal resonator was physically vibrated, while the entire disk-shaped quartz crystal resonator was physically vibrated, which resulted in complex oscillation modes, and thus loss of vibration energy.

As such, it is shown that the convex quartz crystal resonator has greater stability of resonant frequency change and response sensitivity than the disk-shaped quartz crystal resonator.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. An odor sensor, comprising:
at least one convex quartz crystal resonator having a first surface and a second surface;
a pair of first electrodes disposed on the first surface;
a second electrode disposed on the second surface; and
at least one odor-sensitive material disposed on the second electrode.

2. The odor sensor of claim 1, wherein the at least one convex quartz crystal resonator includes a plano-convex quartz crystal resonator, the first surface is a convex-shaped surface, and the second surface is a substantially planar surface.

3. The odor sensor of claim 1, wherein the pair of first electrodes and the second electrode are made of gold.

4. The odor sensor of claim 1, wherein the pair of first electrodes and the second electrode are aligned with a convex-shaped portion of the at least one convex quartz crystal resonator.

5. The odor sensor of claim 1, wherein the at least one odor-sensitive material has a selective affinity for a chemical to be detected.

6. The odor sensor of claim 1, wherein the at least one odor-sensitive material comprises at least one of polycaprolactone, polystyrene, cycloolefin, or acrylic resin.

7. The odor sensor of claim 1, wherein the at least one convex quartz crystal resonator includes an AT-cut convex quartz crystal resonator.

8. (canceled)

9. A method to fabricate an odor sensor, the method comprising:
providing a quartz crystal substrate;
forming a convex portion in the quartz crystal substrate;
forming a pair of first electrodes on a first surface of the convex portion;
forming a second electrode on a second surface of the convex portion; and
applying at least one odor-sensitive material on the second electrode.

10. The method of claim 9, wherein the forming of the convex portion comprises:
applying a photoresist on a surface of the quartz crystal substrate;
patterning the photoresist on the surface of the quartz crystal substrate;
curing the patterned photoresist; and
etching the quartz crystal substrate and the patterned photoresist to form the convex portion.

11. The method of claim 10, wherein the forming of the convex portion further comprises determining a sectional profile of the convex portion, and
wherein the patterning of the photoresist is performed based at least in part on the determined sectional profile of the convex portion.

12. The method of claim 10, wherein the curing of the patterned photoresist comprises heating the patterned photoresist.

13. The method of claim 10, wherein the etching is performed by reactive ion etching (RIE).

14. The method of claim 10, wherein the etching comprises etching the quartz crystal substrate and the patterned photoresist at different etching rates.

15. The method of claim 9, wherein the forming of the pair of first electrodes comprises:
sputtering gold on the first surface of the convex portion; and
patterning the sputtered gold to form the pair of first electrodes.

16. The method of claim 9, wherein the forming of the second electrode comprises:
sputtering gold on the second surface of the convex portion; and
patterning the sputtered gold to form the second electrode.

17. The method of claim 9, wherein the at least one odor-sensitive material has a selective affinity for a chemical to be detected.

18. The method of claim 9, wherein the at least one odor-sensitive material comprises at least one of polycaprolactone, polystyrene, cycloolefin, and acrylic resin.

19. The method of claim 9, wherein the applying of the at least one odor-sensitive material comprises:
applying on the second electrode a solution including the at least one odor-sensitive material.

20. The method of claim 19, wherein the solution comprises an organic solvent that dissolves the at least one odor-sensitive material.

21. The method of claim 20, wherein the organic solvent includes at least one of acetone, trichloroethylene, and alcohol.

22. The method of claim 9, further comprising:
selecting the at least one odor-sensitive material to be applied based at least in part on a chemical to be detected.

23. The method of claim 9, further comprising:
selecting an amount of the at least one odor-sensitive material to be applied on the second electrode.

24. The method of claim 9, further comprising:
selecting an area of the second electrode on which the at least one odor-sensitive material to be applied.

25-26. (canceled)

27. A method to detect odor using an odor sensor, the method comprising:
exposing at least one odor sensitive material that is disposed on a first electrode to a chemical, the first electrode disposed on a first surface of at least one convex quartz crystal resonator;
measuring a change in a resonating frequency of the at least one convex quartz crystal resonator; and
detecting the chemical based at least in part on the measured change in the resonating frequency of the at least one convex quartz crystal resonator.

28. The method of claim 27, wherein the at least one convex quartz crystal resonator has a second surface, a pair of second electrodes are disposed on the second surface.

29. The method of claim 27, wherein the at least one odor-sensitive material has a selective affinity for the associated chemical.
